(19) United States
(12) Patent Application Publication
(10) Pub. No.: US 2019/0336460 A1
(43) Pub. Date: Nov. 7, 2019

(54) COMPOSITION COMPRISING FORMIC ACID OR PHARMACEUTICALLY ACCEPTABLE SALT THEREOF AS ACTIVE INGREDIENT FOR PREVENTING OR TREATING OBESITY OR METABOLIC SYNDROMES CAUSED BY OBESITY

(71) Applicant: KOREA FOOD RESEARCH INSTITUTE, Wanju-gun Jeollabuk-do (KR)

(72) Inventors: Myung Ki LEE, Gyeonggi-do (KR); Sang Dong LIM, Gyeonggi-do (KR); Sung Hun YI, Daejeon (KR); Yong Sun CHO, Seoul (KR); Young Do NAM, Gyeonggi-do (KR); Jin Ju BAE, Daejeon (KR)

(21) Appl. No.: 16/462,909

(22) PCT Filed: Nov. 21, 2017
(86) PCT No.: PCT/KR2017/013277

§ 371 Date: May 21, 2019

(30) Foreign Application Priority Data
Nov. 21, 2016 (KR) 10-2016-0154963

(51) Int. Cl. A61K 31/19; A23L 33/10; A61P 3/04

(52) U.S. Cl. CPC A61K 31/19; A61P 3/04; A23L 33/10

(57) ABSTRACT

The present invention relates to a pharmaceutical composition comprising formic acid or a pharmaceutically acceptable salt thereof as an active ingredient for preventing or treating obesity or metabolic syndromes; and to a food composition and a health functional food each comprising the active ingredient. The formic acid or the salt thereof according to the present invention not only has effects of reducing body weight and inhibiting fat accumulation in organs, but also has activity to effectively lower blood triglyceride and cholesterol levels, and therefore, the composition comprising the formic acid or the salt thereof as an active ingredient can be favorably used as a composition capable of preventing/alleviating or treating obesity or metabolic syndromes. Therefore, the formic acid or the salt thereof according to the present invention can be favorably used as a material for a medical product or health food.

### TECHNICAL FIELD

The present disclosure relates to a composition containing formic acid or a pharmaceutically acceptable salt thereof as an active ingredient for preventing or treating obesity or metabolic syndrome caused by obesity, and to a food composition and a health functional food each containing the active ingredient.

### BACKGROUND ART

Recently, the improvement of living standards due to economic development has enhanced the hygiene environment, but frequent intakes of instant foods and changes of dietary life into meat-based consumption cause intake of excessive calories. However, such changes in the dietary life of modern people has resulted in a fast increase tendency of obese population since the amount of calories consumed is small due to vastly insufficient amount of exercise. Obesity not only causes external problems, but has also been reported to cause several diseases, that is, breast cancer, uterine cancer, and colorectal cancer as well as adult diseases, such as hypertension, diabetes, hyperlipidemia, and coronary artery disease, due to the persistence of obesity, and thus obesity is now treated as one of the deadly diseases [J. Biol. Chem., 273, 32487-32490 (1998); and Nature, 404, 652-660 (2000)].

Obesity has increased by 75% globally since 1980, and in the United States, 33% and 26% of the population is reported to be overweight and obese, respectively (Ahn I S, Park K Y, Do M S. 2007. Weight control mechanisms and antiobesity functional agents. J Korean Soc Food Sci Nutr 36: 503-513). In Korea, the obesity population is also steadily increasing, and according to the 2007 National Health and Nutrition Examination Survey, the obese population has rapidly increased from 26.3% in 1998 to 31.7% in 2005.

This obesity is caused by the imbalance of energy intake and consumption, and the excess energy is converted into a form of fat cells and stored in the body, resulting in increases in the number and size of fat cells. Free fatty acids, cytokines, and the like, which are secreted from accumulated fat cells, cause insulin resistance and increase the inflammatory response, leading to direct causes of chronic disease, such as metabolic syndrome, diabetes, cardiovascular disease, and cancer. In order to treat such obesity, the improvement of eating habits and lifestyle through exercise and diet therapy and the treatment through drug therapy and surgery are introduced, and out of these, as for the development of anti-obesity drugs to suppress obesity, more than 100 kinds of medicines are being sold or under development in the United States, and the market scale therefor is expected to gradually grow in size.

Current medicines for treating obesity may be largely classified into drugs that act on the central nervous system to affect appetite and drugs that act on the gastrointestinal tract to inhibit absorption. The drugs acting on the central nervous system include, depending on the mechanism of each drug, those inhibiting serotonin (5-HT) in the nervous system, such as fenfluramine and dexfenfluramine, those acting on noradrenaline in the nervous system, such as ephedrine and caffeine, and those acting on both serotonin and noradrenaline in the nervous systems to inhibit obesity, such as sibutramine, and these drugs are available in the market. Besides, a representative example of the drugs acting on the gastrointestinal tract to inhibit obesity is orlistat, which has recently been approved as an obesity drug since orlistat inhibits lipase produced in the pancreas to reduce the absorption of fat.

However, out of the conventionally used drugs, fenfluramine or the like causes sides effects, such as primary pulmonary hypertension or cardiac valve lesion, and thus the use thereof has been recently prohibited, sibutramine causes side effects of increasing blood pressure, and orlistat has been reported to have side effects, such as digestive problems, fatty stools, fecal incontinence, and the inhibition of absorption of fat-soluble vitamins. Moreover, other chemically synthetic drugs cause problems, such as reduction in blood pressure or lactic acidosis, and thus could not be used for patients with cardiac insufficiency or kidney disease.

Therefore, there has been recently a growing interest in obesity medicines or anti-obesity health functional foods of natural substances having an action of inhibiting the accumulation of body fat, such as naturally-derived compounds, in order to discover an obesity treatment and prevention method causing fewer side effects and having better effects.

Meanwhile, metabolic syndrome refers to a syndrome that shows risk factors together, such as hypertriglyceridemia, hypertension, hyperglycemia, abnormal blood coagulation, and obesity. The symptoms per se are not fatal, but are likely to develop severe diseases, such as diabetes or ischemic cardiovascular diseases, resulting in a great threat to modern people.

The factors known in association with the causes and treatment of metabolic syndrome are exercise, dietary habits, weight, blood glucose, triglyceride, cholesterol, insulin resistance, adiponectin, leptin, AMPK activity, sex hormones such as estrogen, genetic factors, and the malonyl-CoA concentration in the body, and these are directly or indirectly involved therein.

It is known that the best ways to alleviate these symptoms of metabolic syndrome are exercise, diet restriction, and weight loss. The common denominator in such methods having an effect on metabolic syndrome is to promote energy metabolism to consume the excess energy in the body and thus prevent the accumulation of energy. Due to the lack of exercise relative to the intake of high calorie energy, such as through processed foods and fast foods, excess energy is accumulated as fat, causing various diseases including metabolic diseases. The effective removal of such excess energy is considered to be a method for treatment of metabolic diseases. It is considered to be essential to increase metabolic activity in order to effectively remove excess energy, and for this purpose, it is considered to be necessary to activate the factors involved in the inhibition of lipogenesis, the inhibition of glucogenesis, the stimulation of glucose consumption, the promotion of lipid oxidation, and the production promotion and activation of mitochondria, the core of energy metabolism.

Therefore, the present inventors, during the search for a substance causing no side effects on the living body and having excellent anti-obesity activity, analyzed various organic acids, which are differently contained in the feces between a normal group and an obesity group both of Koreans in their 50s, and tested for anti-obesity pharmaceutical effects thereof. As a result, the present inventors confirmed that experimental groups, in which obesity animal models were orally administered with, especially, formate, had effects of reducing body weight and inhibiting fat accumulation in organs and effectively lowered blood triglyceride and cholesterol levels, and thus completed the present disclosure.

### DETAILED DESCRIPTION OF THE INVENTION

### Technical Problem

Therefore, an aspect of the present disclosure is to provide a pharmaceutical composition for preventing or treating metabolic syndrome, the pharmaceutical composition being capable of inhibiting the accumulation of body fat and effectively lowering blood fat and cholesterol levels.

Another aspect of the present disclosure is to provide a food composition for preventing or alleviating metabolic syndrome, the food composition being capable of inhibiting the accumulation of body fat and effectively lowering blood fat and cholesterol levels.

Still another aspect of the present disclosure is to provide a health functional food for preventing or alleviating metabolic syndrome, the health functional food being capable of inhibiting the accumulation of body fat and effectively lowering blood fat and cholesterol levels.

### Technical Solution

In accordance with an aspect of the present disclosure, there is provided a pharmaceutical composition for preventing or treating metabolic syndrome, the pharmaceutical composition containing, as an active ingredient, formic acid or a pharmaceutically acceptable salt thereof.

The metabolic syndrome may be selected from the group consisting of obesity, diabetes, arteriosclerosis, hypertension, hyperlipidemia, liver diseases, stroke, myocardial infarction, ischemic diseases, and cardiovascular diseases.

The formic acid or pharmaceutically acceptable salt thereof may be contained in 0.1-99 wt % relative to a total weight of the composition.

In accordance with another aspect of the present disclosure, there is provided a food composition for preventing or alleviating metabolic syndrome, the food composition containing, as an active ingredient, formic acid or a pharmaceutically acceptable salt thereof.

In accordance with still another aspect of the present disclosure, there is provided a health functional food for preventing or alleviating metabolic syndrome, the health functional food containing, as an active ingredient, formic acid or a pharmaceutically acceptable salt thereof.

The metabolic syndrome may be selected from the group consisting of obesity, diabetes, arteriosclerosis, hypertension, hyperlipidemia, liver diseases, stroke, myocardial infarction, ischemic diseases, and cardiovascular diseases.

The food may be selected from the group consisting of drinks, meats, chocolates, foods, confectionery, pizzas, ramen, other noodles, gums, candies, ice creams, alcohol drinks, vitamin complexes, and health supplement foods.

### Advantageous Effects

The formic acid or salt thereof according to the present disclosure has not only effects of reducing body weight and inhibiting fat accumulation in organs, but also activity to effectively lower blood triglyceride and cholesterol levels, and thus the composition containing the same as an active ingredient can be helpfully used as a composition capable of preventing/alleviating or treating obesity or metabolic syndrome. Therefore, the formic acid or salt thereof according to the present disclosure can be helpfully used as a substance for medical products or health foods.

### BRIEF DESCRIPTION OF THE DRAWINGS

FIG. 1 is a graph illustrating changes in body weight over the rearing time according to the feeding of various organic acids to high-fat diet-induced obesity experimental animals (A: normal diet group, B: high-fat diet group, C: high-fat diet+formate feeding group, D: high-fat diet+lactate feeding group, E: high-fat diet+propionate feeding group, F: high-fat diet+acetate feeding group).

FIG. 2 shows images illustrating sizes of epididymal fat tissue cells according to the feeding of various organic acids to high-fat diet-induced obesity experimental animals (A: normal diet group, B: high-fat diet group, C: high-fat diet+formate feeding group, D: high-fat diet+lactate feeding group, E: high-fat diet+propionate feeding group, F: high-fat diet+acetate feeding group).

### BEST MODE FOR CARRYING OUT THE INVENTION

Hereinafter, the terms used herein will be described.

As used herein, the term “pharmaceutical acceptable” refers to not inhibiting biological activity and characteristics of an administered active substance without greatly stimulating an organism.

As used herein, the term “prevention” refers to all the activities that inhibit symptoms or delay the progression of a particular disease (e. g., obesity or metabolic syndrome) by the administration of the composition of the present disclosure.

As used herein, the term “treatment” refers to all the activities that alleviate or favorably modify symptoms of a particular disease (e. g., obesity or metabolic syndrome) by the administration of the composition of the present disclosure.

As used herein, the term “alleviation” refers to all the activities that at least reduce parameters associated with a condition to be treated, for example, the severity of symptoms.

As used herein, the term “administration” refers to providing an individual with a predetermined composition of the present disclosure through any appropriate method. The individual refers to any animal having a particular disease, of which symptoms can be alleviated by the administration of the composition of the present disclosure, such as a human being, a monkey, a dog, a goat, a pig, or a mouse.

As used herein, the term “pharmaceutically effective amount” refers to an amount sufficient to treat a disease at a reasonable benefit or risk ratio, which is applicable to medical treatment. The pharmaceutically effective amount may be determined depending on factors including the disease type and severity of an individual, activity of drugs, sensitivity to drugs, time of administration, route of administration, excretion ratio, duration of treatment, and concurrently used drugs, and other factors well-known in the medical field.

Hereinafter, the present disclosure will be described in detail.

The present disclosure is characterized by providing a composition containing formic acid or a pharmaceutically acceptable salt thereof as an active ingredient for preventing or treating metabolic syndrome.

As cited herein, the “formic acid” is also called ant-acid or hydrogen carboxylic acid. The chemical formula is HCOOH. Formic acid has a molecular weight of 46.0, which is the smallest in carboxylic acids, and has a boiling point of 100.5° C., a melting point of 8.4° C., and a specific gravity of 1.220. Such formic acid is not only present in nature but also obtained from synthesis and is a fluidic and colorless liquid. The formic acid is slightly fumed when exposed to air and has irritating odor and corrosiveness. Formic acid is used in dyeing, tanning, coagulation of latex, or as a preservative in medicines or organic synthesis.

The formic acid according to the present disclosure may be used in the form of a salt, preferably a pharmaceutically acceptable salt. For example, the formic acid may be used in the form of calcium formate or sodium formate.

Calcium formate has a chemical formula of C₂H₂CaO₄ and a molecular weight of 130.1154, is present as white crystals or powder, and has hygroscopic properties and a slightly bitter taste. Calcium formate is neutral, nontoxic, and soluble in water. Such calcium formate can be used as a feed additive, and is known to have oxidative, antifungal, and antimicrobial actions when applied to various animals.

Sodium formate has a chemical formula of NaHCO₂ and a molecular weight of 68.01, is present as a deliquescent crystalline white powder, has a melting point of 253° C., and is degraded into hydrogen and sodium oxalate when heated at a high temperature. Sodium formate is used for medical products, cosmetic products, and the like, and is also used in organic synthesis. Sodium formate is low toxic when used in the human body, and is known to cause no side effect up to 10 g when administered orally (SODIUM FORMATE—National Library of Medicine HSDB Database, https://toxnet.nlm.nih.gov/cgi-bin/sis/search/a?dbs+hsdb:@term+@DOCNO+744).

The examples below of the present disclosure confirmed effects of reducing body weight, inhibiting fat accumulation in organ tissues, and lowering blood triglyceride and cholesterol levels according to the oral administration of sodium formate into obesity-induced animal models, and also, first confirmed that the size of epididymal fat cells was observed to be small in the sodium formate administration group.

Therefore, the present disclosure provides a pharmaceutical composition containing formic acid or a pharmaceutically acceptable salt thereof as an active ingredient for preventing or treating metabolic syndrome.

As used herein, the “metabolic syndrome” may be a metabolic disease caused by obesity or a metabolic disease caused by any other cause. Examples of the metabolic syndrome may be selected from the group consisting of obesity, diabetes, arteriosclerosis, hypertension, hyperlipidemia, liver diseases, stroke, myocardial infarction, ischemic diseases, and cardiovascular diseases, but are not limited thereto.

As used herein, the “obesity” includes simple obesity, symptomatic obesity, child obesity, adult obesity, hyperplasia obesity, hypertrophic obesity, upper body obesity, lower body obesity, visceral fat obesity, and subcutaneous fat obesity, but is not limited thereto.

The pharmaceutical composition of the present disclosure may be prepared by using a pharmaceutically acceptable and physiologically acceptable adjuvant in addition to the above-mentioned active ingredient, and examples of the adjuvant may be an excipient, a disintegrant, a sweetener, a binder, a coating agent, a swelling agent, a lubricant, a glydent, a flavoring agent, or the like.

The pharmaceutical composition, for administration, may be preferably formulated into a pharmaceutical composition by further containing at least one pharmaceutically acceptable carrier, in addition to the foregoing active ingredient.

The formulation form of the pharmaceutical composition may be granules, a powder, a tablet, a coated tablet, a capsule, a suppository, a liquid, syrup, juice, a suspension, an emulsion, drops, an injectable liquid, or the like. For example, for the formulation into a tablet or capsule, the active ingredient may be combined with an oral, non-toxic, pharmaceutically acceptable inert carrier, such as ethanol, glycerol, and water. Also, if desired or necessary, a suitable binder, a lubricant, a disintegrant, and a coloring agent may also be contained in the form of being mixed. The suitable binder includes, but is not limited to, natural sugars, such as starch, gelatin, glucose, or beta-lactose, corn sweeteners, natural and synthetic gums, such as acacia, tragacanth, or sodium oleate, sodium stearate, magnesium stearate, sodium benzoate, sodium acetate, sodium chloride, and the like. The disintegrant includes, but is not limited to, starch, methyl cellulose, agar, bentonite, xanthan gum, and the like. As for the composition formulated into a liquid solution, the pharmaceutically acceptable carrier is sterilizable and biocompatible, and may include saline, sterile water, Ringer's solution, buffered saline, an albumin injection solution, a dextrose solution, a maltodextrin solution, glycerol, ethanol, and a mixture of one or more thereof, and if necessary, other ordinary additives, such as an antioxidant, a buffer solution, and a bacteriostatic agent, may be added. In addition, the composition may be formulated into an injectable formulation, such as an aqueous solution, a suspension, or an emulsion, a pill, a capsule, granules, or a tablet by further adding to a diluent, a dispersant, a surfactant, a binder, and a lubricant thereto. Furthermore, the pharmaceutical composition may be preferably formulated according to the disease or ingredient by a suitable method in the art or using a method disclosed in Remington's Pharmaceutical Science, Mack Publishing Company, Easton Pa.

In an embodiment of the present disclosure, the formic acid or pharmaceutically acceptable salt thereof of the present disclosure may be contained in 0.1-99 wt % relative to a total weight of the composition.

The composition of the present disclosure may be used in a mixture with an anti-obesity agent, which has been conventionally used, in addition to the foregoing formic acid or pharmaceutically acceptable salt thereof, so long as the anti-obesity agent reacts with the formic acid (or salt thereof) to cause no side effect.

Furthermore, the present disclosure provides a food composition containing formic acid or a pharmaceutically acceptable salt thereof as an active ingredient for preventing or alleviating metabolic syndrome.

As used herein, the “metabolic syndrome” may be a metabolic disease caused by obesity or a metabolic disease caused by any other cause. Examples of the metabolic syndrome may be selected from the group consisting of obesity, diabetes, arteriosclerosis, hypertension, hyperlipidemia, liver diseases, stroke, myocardial infarction, ischemic diseases, and cardiovascular diseases, but are not limited thereto.

In an embodiment of the present disclosure, the formic acid or pharmaceutically acceptable salt thereof of the present disclosure may be contained in 0.1-99 wt % relative to a total weight of the food composition. For reference, the Korean Food and Drug Administration states that the daily allowable intake of formic acid is 0-3 mg/kg bw.

The food composition of the present disclosure, like ordinary food compositions, may contain various flavoring agents or natural carbohydrates as additional ingredients, in addition to formic acid or a pharmaceutically acceptable salt thereof as an active ingredient.

Examples of the foregoing natural carbohydrates may include ordinary sugars, such as monosaccharides (e.g., glucose and fructose); disaccharides (e.g., maltose and sucrose), and polysaccharides (e.g., dextrin and cyclodextrin); and sugar alcohols, such as xylitol, sorbitol, and erythritol. As the foregoing flavoring agents, natural flavoring agents (thaumatin), and stevia extracts (e.g., rebaudioside A, glycyrrhizin, etc.), and synthetic flavoring agents (saccharin, aspartame, etc.) may be advantageously used.

The food composition of the present disclosure may be preferably formulated into a food composition by further containing at least one sitologically acceptable or pharmaceutically acceptable carrier, in addition to the foregoing active ingredient.

The formulation form of the food composition may be a tablet, a capsule, a powder, granules, a liquid, a pill, a liquid preparation, syrup, juice, a suspension, an emulsion, drops, or the like. For example, for the formulation into a tablet or capsule, the active ingredient may be combined with an oral, non-toxic, pharmaceutically acceptable inert carrier, such as ethanol, glycerol, and water. Also, if desired or necessary, a suitable binder, a lubricant, a disintegrant, and a coloring agent may also be contained in the form of being mixed. The suitable binder includes, but is not limited to, natural sugars, such as starch, gelatin, glucose, or beta-lactose, corn sweeteners, natural and synthetic gums, such as acacia, tragacanth, or sodium oleate, sodium stearate, magnesium stearate, sodium benzoate, sodium acetate, sodium chloride, and the like. The disintegrant include, but is not limited to, starch, methyl cellulose, agar, bentonite, xanthan gum, and the like. As for the composition formulated into a liquid solution, the pharmaceutically acceptable carrier is sterilizable and biocompatible, and may include saline, sterile water, Ringer's solution, buffered saline, an albumin injection solution, a dextrose solution, a maltodextrin solution, glycerol, ethanol, and a mixture of one or more thereof, and if necessary, other ordinary additives, such as an antioxidant, a buffer solution, and a bacteriostatic agent, may be added. In addition, the food composition may be formulated into an injectable formulation, such as an aqueous solution, a suspension, or an emulsion, a pill, a capsule, granules, or a tablet by further adding to a diluent, a dispersant, a surfactant, a binder, and a lubricant thereto.

The food composition of the present disclosure formulated in the above-described manners can be used as a functional food or added to various foods.

Exemplary foods to which the composition of the present disclosure is addable may include, for example, drinks, meats, chocolates, foods, confectionery, pizzas, ramen, other noodles, gums, candies, ice creams, alcohol drinks, vitamin complexes, health supplement foods, and the like.

In addition, the food composition may contain, in addition to formic acid or a pharmaceutically acceptable salt thereof as an active ingredient, various nutrients, vitamins, minerals (electrolytes), flavoring agents, such as synthetic flavoring agents and natural flavoring agents, coloring agents, extenders (cheese, chocolate, etc.), pectic acid and salts thereof, alginic acid and salts thereof, organic acids, protective colloidal thickeners, pH adjusters, stabilizers, preservatives, glycerin, alcohols, carbonating agents used for carbonated drinks, and the like. Besides, the food composition of the present disclosure may contain fruit flesh for manufacturing natural fruit juice, fruit juice drinks, and vegetable drinks.

The formic acid or pharmaceutically acceptable salt thereof as an active ingredient of the present disclosure is a kind of organic acid, and has few side effects like chemicals, and thus can be safely used for long-term taking for the purpose of imparting functionality, such as alleviation of anti-obesity or metabolic syndrome.

Furthermore, the present disclosure provides a health functional food containing formic acid or a pharmaceutically acceptable salt thereof as an active ingredient for preventing or alleviating metabolic syndrome.

The health functional food of the present disclosure may be manufactured and processed in the form of a tablet, a capsule, a powder, granules, a liquid, a pill, or the like, for the purpose of prevention or alleviation of obesity or metabolic syndrome.

As used herein, the term “health functional food” refers to a food that is manufactured and processed by using raw materials or ingredients having functionality useful to the human body according to the Korean Health/Functional Food Act, and means one that is taken for the purpose of obtaining effects useful for regulating nutrients for the structure and function of the human body or for obtaining effects useful for hygienic purposes such as a physiological action.

The health functional food of the present disclosure may contain ordinary food additives, and the suitability thereof as a food additive, unless otherwise specified, is determined by the standards and criteria for a corresponding item according to the general provisions of Korean Food Additives Codex and General Test Method approved by the Korean Food and Drug Administration.

Examples of the items listed on the “Korean Food Additives Codex” may include: chemical synthetic products, such as ketone, glycine, potassium citrate, nicotinic acid, and cinnamic acid; natural additives, such as persimmon color, a licorice root extract, crystalline cellulose, Kaoliang color, and guar gum; and mixed preparations, such as sodium L-glutamate preparations, an alkali agent for noodles, a preservative preparation, and a tar color formulation.

For example, as for a tablet form of health functional food, a mixture obtained by mixing the formic acid or pharmaceutically acceptable salt thereof as an active ingredient of the present disclosure with an excipient, a binder, a disintegrant, and other additives is granulated by an ordinary method, and then a glydent and the like is added thereto, followed by compressive molding, or the mixture is directly compressive molded. The tablet form of health functional food may contain a sour taste agent and the like as necessary.

Out of the capsule form of health functional foods, a hard capsule may be manufactured by filling an ordinary hard capsule with the formic acid or pharmaceutically acceptable salt thereof as an active ingredient of the present disclosure, and a soft capsule may be manufactured by filing a capsule, such as gelatin, with a mixture obtained by mixing the formic acid and an additive, such as an excipient. The soft capsule may contain a plasticizer, a coloring agent, a preservative, or the like, such as glycerin or sorbitol, as necessary.

A pill form of health functional food may be manufactured by molding a mixture of the formic acid or pharmaceutically acceptable salt thereof as an active ingredient of the present disclosure with an excipient, a binder, a disintegrant, and the like through an existing known method, and as necessary, the pill form of health functional food may be coated with white sugar or another coating agent, or a surface thereof may be coated with a material, such as starch or talc.

A granule form of health functional food may be manufactured by granulating a mixture of the formic acid or pharmaceutically acceptable salt thereof of the present disclosure with an excipient, a binder, a disintegrant, and the like through an existing known method, and the granule form of health functional food may contain a coloring agent, a sour taste agent, or the like, as necessary.

The health functional food may include drinks, meats, chocolates, foods, confectionery, pizzas, ramen, other noodles, gums, candies, ice creams, alcohol drinks, vitamin complexes, and health supplement foods.

Hereinafter, the present disclosure will be described in detail with reference to examples. However, these examples are given for specifically illustrating the present disclosure, and the scope of the present disclosure is not limited thereto.

### MODE FOR CARRYING OUT THE INVENTION

### Examples

Statistics

For statistical analysis of fecal organic acids in an obesity group and a normal control group, the system Statistical Package for Social Science (SPSS, SPSS Inc., Chicago, Ill., USA) software package (version 12.0) was used, and a significant difference between samples was analyzed by T-test at a level of p<0.05. For statistical analysis of body weight, organ weights, and blood, a significant difference between samples was analyzed by Duncan's multiple range test at a level of p<0.05.

### Example 1

Collection of Feces and Analysis of Fecal Organic Acids in Normal Control Group and Obesity Group

The feces of a normal control group (n=33) and an obesity group (n=44) both of males in their 50s used in this study were samples that had been obtained from the Catholic University, Seoul Catholic Hospital (Seocho, Korea) through IRB deliberation (IRB acquisition at Seoul Catholic Hospital: KC14TIS10325). Analysis of organic acids in the feces were conducted by the National Instrumentation Center for Environmental Management (NICEM, Korea), Seoul National University. For analysis of organic acids, each sample was 10-fold diluted with tertiary distilled water, centrifuged at 3000 rpm for 10 min, and then subjected to filtration using a 0.22 μm-membrane filter, and thus was used as a sample for HPLC (Ultimate3000, Dionex, USA) analysis. The HPLC column used was an Aminex 87H column (300×7.8 mm), and the temperature was maintained at 40° C. The mobile phase was allowed to flow at a rate of 0.5 ml/min using 0.01 N H₂SO₄. A single injection of the sample was 10 μl, and was analyzed for 30 min using the detector RI (Shodex RI-101, Japan) and UV (210 nm).

As a result, as shown in Table 1 below, the organic acids detected commonly in normal people and obese people were formic acid, acetic acid, and butyric acid. The content of formic acid was 0.22 mMol in the normal control group, which was 2.4-fold higher compared with the obesity group, indicating a significant difference. The contents of acetic acid and butyric acid in the normal control group were 0.9-fold and 0.8-fold compared with the obesity group, respectively.

Meanwhile, such content differences were obtained from the analysis results including the patients with diabetes, obesity, hypertension, and hyperlipidemia, greatly associated with obesity, and thus analysis was again conducted excluding these patients.

As a result, as shown in Table 2 below, the formic acid was 0.27 mMol in the normal control group, which was about 6.8-fold higher compared with the obesity group, indicating a significant difference. Whereas, the contents of acetic acid and butyric acid in the normal control group were 0.82-fold and 0.66-fold compared with the obesity group, respectively, but such data indicated no significant difference, and propionic acid was detected in neither the normal control group nor the obesity group.

TABLE 1
Comparison of average values of fecal organic acids
between obesity group and normal control group

| | Organic acid content (mMol) | | | |
|---|---|---|---|---|
| | | Lactic | | Butyric |
| Sample | Formic acid | acid | Acetic acid | acid |
| Normal | 0.22 ± 2.29^{1)A2)} | ND³⁾ | 8.72 ± 3.76^{a} | 2.17 ± 1.49^{a} |
| control | | | | |
| group | | | | |
| [N = 33] | | | | |
| Obesity | 0.09 ± 0.14^{B} | ND | 9.98 ± 2.69^{a} | 2.88 ± 1.89^{a} |
| group | | | | |
| [N = 44] | | | | |

¹⁾Each value represents the mean ± S.D for groups
²⁾Means followed by the same letter in the column are not significantly different (p < 0.05)
³⁾ND: not detected

TABLE 2
Comparison of average values of fecal organic acids
between obesity group and normal control group excluding disease
patients (any one of obesity, hypertension, and hyperlipidemia

| | Organic acid content (mMol) | | | |
|---|---|---|---|---|
| | | Lactic | | Butyric |
| Sample | Formic acid | acid | Acetic acid | acid |
| Normal | 0.27 ± 0.31^{1)A2)} | ND³⁾ | 8.72 ± 3.73^{A} | 2.30 ± 1.59^{A} |
| control | | | | |
| group | | | | |
| [N = 23] | | | | |
| Obesity | 0.04 ± 0.09^{B} | ND | 10.60 ± 2.58^{A} | 3.46 ± 2.42^{A} |
| group | | | | |
| [N = 21] | | | | |

¹⁾Each value represents the mean ± S.D for groups
²⁾Means followed by the same letter in the column are not significantly different (p < 0.05)
³⁾ND: not detected

It was confirmed through the above results that out of the organic acids detected in feces, only formic acid was significantly higher in the normal control group compared with the obesity group. Therefore, formic acid was thought to have an anti-obesity effect in the intestinal environment.

For reference, the results of the present disclosure as described above are meaningful in that the results were derived from the feces of Korean males in their 50s. The reason is that the conventional obesity studies were conducted not on Korean-customization, but on other races or ethnic groups, and also, as for the target age for obesity studies, the results optimized for adult obesity could not be deduced when obesity studies were not conducted on adults (obesity studies were conducted on infants and adolescents). The human physiology is different between the adolescence and menopause, and it is well known that the fecal bacteria flora is also greatly different between children and adults, and thus it is obvious that the resultant fecal metabolites are different therebetween.

Hence, it was meaningful in that for the conduction of research optimized for Korean adult obesity, the present inventors examined differences between normal adults and obese adults in Korean adults in their 50s through fecal collection and organic acid analysis, and therefore first discovered a factor having a direct correlation with Korean adult obesity.

Accordingly, animal experiments were further conducted in order to investigate effects of formic acid and the other organic acids on obesity as below.

### Example 2

Preparation of Obesity Animal Models

<2-1> Experimental Animals and Diet

In the present experiment, five-week-old C57BL/6J mice were purchased from Saeron Bio (Uiwang, Korea), and acclimated for 1 week before use in the experiment. The rearing room was controlled to have a temperature of 20±2° C., a humidity of 55±10%, and a light/darkness cycle of 12 hours during the experimental period. The experimental animals were divided into 6 groups according to the randomized block design, after 1-week of normal diet. The experimental groups were classified into Group A (normal diet group; n=10), Group B (high-fat diet control group; n=10), Group C (high-fat diet+sodium formate 10 mmol; n=9), Group D (high-fat diet+sodium butyrate 10 mmol; n=9), Group E (high-fat diet+sodium propionate 10 mmol; n=8), and Group F (high-fat diet+sodium acetate 10 mmol; n=4).

<2-2> Treatment of Experimental Animals

Water and feed were freely accessible. Groups A and B were orally administered with physiological saline, and the other groups were orally administered with corresponding organic acids once a day (2 μg/g bodyweight). For the test groups, obesity was induced by a high-fat diet for 13 weeks, and a high-fat diet (D12492; 60% of the calories) purchased from Research Diet (USA) was used in the experiment. All animal experiments in the present study were conducted under the approval (KFRI-M-16043(263)) of the Institutional Animal Care and Use Committee (IACUC) of the Korea Food Research Institute.

### Example 3

Body and Organ Weight Measurement and Blood Analysis in Experimental Animals

The body weights of the experimental animals were measured at a fixed time once a week during the experiment. The experimental animals were fasted for 12 hours before sacrifice, and blood was collected from eyes of the experimental animals anesthetized with ether. In order to prevent the coagulation of the blood after collection, the blood was placed in anti-coagulation tubes and left in an ice bath for 20 min. The collected blood samples were centrifuged at 3000 rpm for 10 min to separate serum therefrom, and refrigerated until use in the experiment. Thereafter, the kit through an enzymatic method was purchased from Young Dong Diagnostics (Yong-In, Korea) to measure the levels of blood lipids (triglyceride, cholesterol, HDL-Cholesterol, and LDL-Cholesterol). The organs were extracted, washed with physiological saline, dehydrated with a filter bed, and then weighed.

<3-1> Body Weight Change

In the feces of the normal control group (n=33) and the obesity group (n=44) of males in their 50s obtained through IRB deliberation in the present experiment, formic acid was significantly lower in the normal control group than the obesity group, and thus animal experiments associated with organic acids were conducted.

The high-fat diet-induced obesity experimental animals (C57BL/6J mice) were orally administered with each 10 mmol corresponding organic acid for 13 weeks, and the weights of the mice were measured once a week during the rearing.

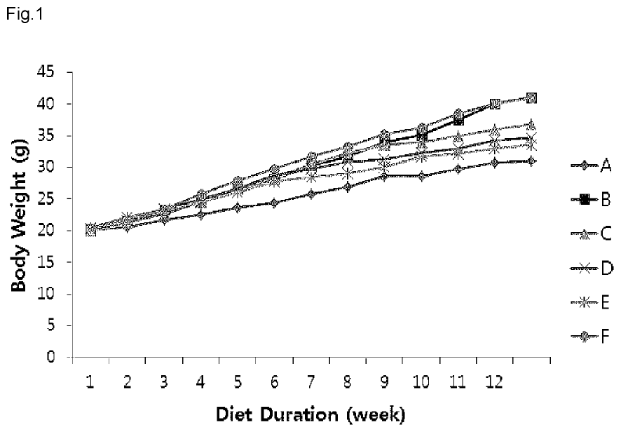

As a result, as shown in FIG. 1, the average body weight after 13 weeks of rearing was 31.00 g in Group A (normal diet control group), 41.09 g in Group B (high-fat diet control group), 36.78 g in Group C (formate oral administration), 31.25 g in Group D (butyrate oral administration), 30.05 g in Group E (propionate oral administration), and 41.08 g in Group E (acetate oral administration), and Groups C, D, and E showed relatively lower values compared with the high-fat diet control group. Compared with the high-fat diet control group, Group F showed no significant difference (p<0.05), but Group C showed a reduction in body weight gain of 10.4%, Group D 15.6%, and Group E 18.3%.

<3-2> Effect on Organ Weights

The kidney fat, epididymal fat, subcutaneous fat, brown fat, prostate, liver, and spleen were extracted immediately after blood collection from the control and experimental groups, and washed with physiological saline, subjected to surface moisture removal, and then weighed. The results are shown in Table 3 below.

The weight of kidney fat was significantly higher in the high-fat diet control group rather than the normal diet control group, and the weights of kidney fat in Groups C, E, and F were 0.72 g, 0.71 g, and 0.66 g, respectively, which were significantly lower compared with the high-fat control diet group (p<0.05). The reason was thought to be that the feeding of organic acids, such as formic acid, propionic acid, and acetic acid, would inhibit the accumulation of kidney fat.

The weight of epididymal fat was observed to be more affected by diet than those of the other types of fat. In addition, all the organic acid treatment groups showed lower values compared with the high-fat diet control group, while group C showed a 19.6% lower value, Group D 20%, Group E 23.6%, and Group F 7.6%, indicating significant differences except for Group F.

The weight of subcutaneous fat was also observed to be greatly affected by diet, while Group C showed 1.08 g, Group D 1.16 g, and Group E 1.07 g. All the organic acid treatment groups showed lower values compared with 1.59 g in the high-fat diet control group. Compared with the high-fat diet control group, Group C showed a 31.16% lower value, Group D 26.58%, and Group E 32.27%. Group F showed a 10.13% higher value, indicating no significant difference. It was thought that the feeding of organic acids excluding acetate would inhibit fat accumulation in epididymal fat and subcutaneous fat.

As for the weight of brown fat, only Group F out of the organic acid treatment groups showed a 22.22% higher value, indicating no significant difference, and therefore, the brown fat was considered to be an organ less affected by diet. The weight of prostate was about 1.9-fold difference between the normal diet control group and the high-fat control diet group, and the prostate was observed to be a tissue more affected by diet. The weight of prostate was higher in all the organic acid treatment groups compared with the high-fat diet control group, indicating no significant difference. The weight of liver and the weight of spleen showed no significant difference among the groups.

TABLE 3
Measurement of weights of mouse organs in each group
(Unit: g)

| | Kidney | Epididymal | Subcutaneous | Brown | | | |
|---|---|---|---|---|---|---|---|
| Group | fat | fat | fat | fat | Prostate | Liver | Spleen |
| A¹⁾ | 0.43 ± 0.14^{2)Da)} | 1.12 ± 0.24^{C} | 0.65 ± 0.16^{C} | 0.09 ± 0.03^{A} | 0.10 ± 0.04^{B} | 0.93 ± 0.12^{AB} | 0.06 ± 0.01^{A} |
| (n = 10) | | | | | | | |
| B | 0.84 ± 0.12^{A} | 2.50 ± 0.48^{A} | 1.58 ± 0.39^{A} | 0.09 ± 0.03^{A} | 0.19 ± 0.07^{A} | 0.96 ± 0.12^{AB} | 0.07 ± 0.01^{A} |
| (n = 10) | | | | | | | |
| C | 0.72 ± 0.08^{BC} | 2.01 ± 0.24^{B} | 1.08 ± 0.21^{B} | 0.09 ± 0.03^{A} | 0.20 ± 0.05^{A} | 0.90 ± 0.10^{AB} | 0.07 ± 0.01^{A} |
| (n = 9) | | | | | | | |
| D | 0.71 ± 0.12^{BC} | 2.00 ± 0.29^{B} | 1.16 ± 0.25^{B} | 0.09 ± 0.04^{A} | 0.24 ± 0.06^{A} | 0.81 ± 0.26^{B} | 0.07 ± 0.01^{A} |
| (n = 9) | | | | | | | |
| E | 0.66 ± 0.09^{C} | 1.91 ± 0.44^{B} | 1.07 ± 0.24^{B} | 0.09 ± 0.02^{A} | 0.21 ± 0.06^{A} | 0.84 ± 0.05^{B} | 0.06 ± 0.01^{A} |
| (n = 8) | | | | | | | |
| F | 0.81 ± 0.07^{BAB} | 2.31 ± 0.45^{AB} | 1.74 ± 0.31^{A} | 0.11 ± 0.03^{A} | 0.22 ± 0.07^{A} | 1.09 ± 0.48^{A} | 0.07 ± 0.01^{A} |
| (n = 4) | | | | | | | |

¹⁾A: normal diet group, B: high fat diet group, C: high fat diet group with formate, D: high fat diet group with butyrate, E: high fat diet group with propionate, F: high fat diet group with acetate.
²⁾Each value represents the mean ± S.D. for groups.
³⁾Means followed by the same letter in the column are not significantly different (p < 0.05)

<3-3> Blood Analysis

The effects of the administration of formate, butyrate, propionate, and acetate of the organic acids on serum lipid concentrations are shown in Table 4 below.

As a result, the serum triglyceride (TG) concentration was 86.64±4.21 mg/dL in the normal diet group (A), but 130.10±7.69 mg/dL in the high-fat diet control group (B), which shows a higher concentration than the normal diet group. The formate administration group (C) showed 99.48±14.18 mg/dL, which was a 23.54% lower value compared with the high-fat diet control group, and the butyrate administration group (D) showed 128.01±14.72 mg/dL, which was a similar level compared with the high-fat diet control group. The propionate administration group (E) showed 120.68±10.07 mg/dL, which was a 7.2% lower value compared with the high-fat diet control group, and the acetate administration group (F) showed 155.21±5.43 mg/dL, which was a significantly higher value compared with the high-fat diet control group. It was thought through these results that the reduction in blood triglyceride concentration was affected by the administration of organic acids excluding acetate.

Meanwhile, the content of cholesterol was increased by 61.2% in the high-fat diet feeding control group compared with the normal diet group (A), and was significantly reduced in the administration of organic acids compared with the high-fat diet feeding control group. The content of cholesterol was reduced by 7.8% in the administration of acetate compared with the high-fat diet feeding group, indicating no significant difference. Out of the organic acid administration groups, the formate administration group reduced the content of cholesterol by 36.6% compared with the high-fat diet control group (B), and thus was thought to have a highest effect on anti-obesity.

HDL-cholesterol, which is an indicator of anti-arteriosclerosis, has a protective action against coronary heart disease by transporting cholesterol from peripheral blood vessels to the liver to deliver arteriosclerosis in a direction that does not progress arteriosclerosis. As for the HDL-cholesterol concentration in the present experiment, the organic acid administration groups excluding acetate showed significantly lower values compared with the high-fat diet group (B), and the acetate administration group showed a similar level compared with the high-fat diet group (B), indicating no significant difference.

LDL-cholesterol is a main delivery type for blood cholesterol. LDL-cholesterol accumulates cholesterol on the arterial vascular walls to accelerate arteriosclerosis, and thus there is a close correlation between the concentration of blood LDL-cholesterol and the occurrence of cardiocirculatory diseases. The content of LDL-cholesterol was 23.62% higher in the high-fat diet control group (B) compared with the normal diet group (A). Out of the organic acid administration groups, the formate and butyrate administration groups showed lower values compared with the high-fat diet control group.

TABLE 4
Measurement of serum lipid concentrations in
mice of each group (Unit: mg/dl)

| Group | Cholesterol | Triglyceride | HDL | LDL |
|---|---|---|---|---|
| A¹⁾ (n = 10) | 77.29 ± 9.69^{2)C3)} | 86.64 ± 4.21^{C} | 52.73 ± 1.19^{C} | 22.27 ± 0.39^{E} |
| B (n = 10) | 124.59 ± 9.45^{A} | 130.10 ± 7.69^{B} | 61.09 ± 1.36^{A} | 27.53 ± 1.69^{C} |
| C (n = 9) | 79.00 ± 2.81^{C} | 99.48 ± 14.18^{C} | 41.51 ± 3.56^{D} | 26.24 ± 0.29^{CD} |
| D (n = 9) | 95.63 ± 3.87^{B} | 128.01 ± 14.72^{B} | 55.99 ± 0.43^{B} | 24.95 ± 1.23^{D} |
| E (n = 8) | 95.63 ± 5.41^{B} | 120.68 ± 10.07^{B} | 50.12 ± 1.69^{C} | 30.41 ± 0.51^{B} |
| F (n = 4) | 114.86 ± 3.76^{A} | 155.21 ± 5.43^{A} | 61.09 ± 2.21^{A} | 32.34 ± 1.44^{A} |

¹⁾A: normal diet group, B: high fat diet group, C: high fat diet group with formate, D: high fat diet group with butyrate, E: high fat diet group with propionate, F: high fat diet group with acetate.
²⁾Each value represents the mean ± S.D. for groups.
³⁾Means followed by the same letter in the column are not significantly different (p < 0.05)

From the above results, the contents of triglyceride and total cholesterol in lipids, which are the causative substances of adult diseases, were reduced by the supply of formate, butyrate, and propionate, and especially, the contents of triglyceride and total cholesterol were significantly reduced in the formate administration group compared with the high-fat diet control group, and thus it was thought that formate will be effective in anti-obesity and the prevention of cardiovascular diseases.

### Example 4

Size Measurement of Epididymal Fat Cells

For size measurement of epididymal fat cells, the epididymal fat tissue extracted according to the method by Hirsch and Gallian (Hirsch, J. and Gallian, E. 1968. Methods for the determination of adipose cell size in man and animals. J. Lipid Res. 9:110-119) was immobilized with 10% formalin, and passed through a 250 μm-nylon filter to remove fibrous tissues and small tissues, and then washed with PBS to completely the tissues. The immobilized tissue was sliced into 18 μm using a cryostat microtome, and fat cells were stained by hematoxylin and eosin (H&E) fat staining. The fat cells were decolorized using 60% isopropanol after the staining, and then imaged using a digital camera under a microscope. For size analysis of fat cells, the area of fat cells was measured using Image J Software (National Institute of Health, Maryland, USA).

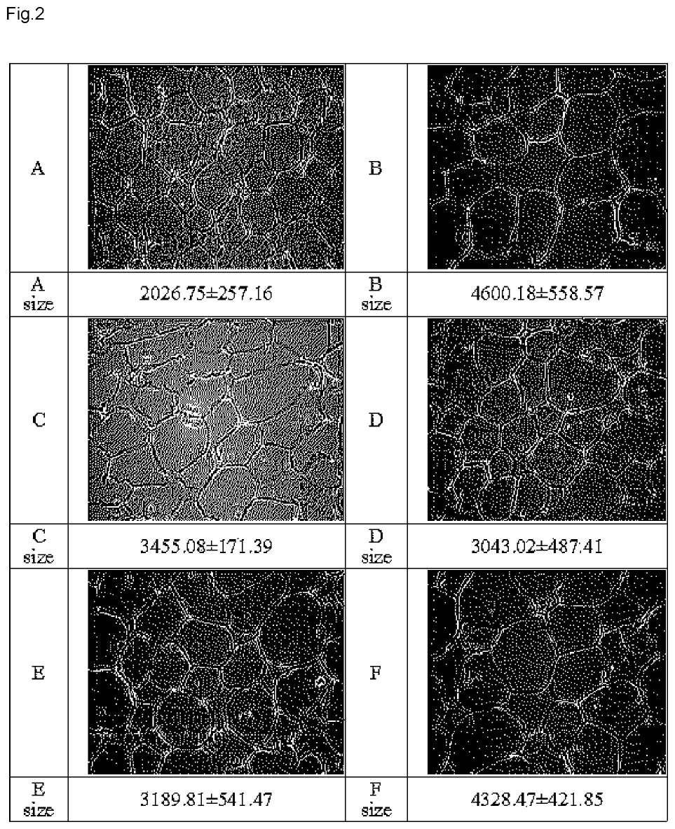

The size measurement results of epididymal fat cells through the present experiment are shown in FIG. 2. From the result of fat cell size distribution, Group B had the largest fat cell size, 4600.18 μm², which were 55.9% higher compared with Group A. The fat cell size was smaller in that order of butyrate, propionate, formate, and acetate administration groups among the organic acid administration groups. Considering that all the organic acid administration groups showed smaller fat cell sizes compared with the high-fat diet control group, it was thought that the feeding of organic acids would inhibit the accumulation of fat cells caused by high-fat diet. When the fat cells stained with H&E were observed under a microscope, the differences in fat cell size could also be seen by the naked eyes.

As examined above, formate was observed to have an anti-obesity effect, and it would be better to expect formate-related anti-obesity effects in Koreans.

As set forth above, the present disclosure has been described with reference to preferable embodiments. Those skilled in the art to which the present disclosure pertain would understand that the present disclosure could be implemented in a modified form without departing from the inherent characteristics of the present disclosure. Accordingly, the embodiments described herein should be considered from an illustrative aspect rather than from a restrictive aspect. The scope of the present disclosure should be defined not by the detailed description but by the appended claims, and all differences falling within a scope equivalent to the claims should be construed as being included in the present disclosure.

### INDUSTRIAL APPLICABILITY

The formic acid or salt thereof according to the present disclosure can be helpfully used as a substance for medical products or health foods.

1. A pharmaceutical composition for preventing or treating metabolic syndrome, the pharmaceutical composition containing, as an active ingredient, formic acid or a pharmaceutically acceptable salt thereof.

2. The pharmaceutical composition of claim 1, wherein the metabolic syndrome is selected from the group consisting of obesity, diabetes, arteriosclerosis, hypertension, hyperlipidemia, liver diseases, stroke, myocardial infarction, ischemic diseases, and cardiovascular diseases.

3. The pharmaceutical composition of claim 1, wherein the formic acid or pharmaceutically acceptable salt thereof is contained in 0.1-99 wt % relative to a total weight of the composition.

4. A food composition for preventing or alleviating metabolic syndrome, the food composition containing, as an active ingredient, formic acid or a pharmaceutically acceptable salt thereof.

5. A health functional food for preventing or alleviating metabolic syndrome, the health functional food containing, as an active ingredient, formic acid or a pharmaceutically acceptable salt thereof.

6. The health functional food of claim 5, wherein the metabolic syndrome is selected from the group consisting of obesity, diabetes, arteriosclerosis, hypertension, hyperlipidemia, liver diseases, stroke, myocardial infarction, ischemic diseases, and cardiovascular diseases.

7. The health functional food of claim 5, wherein the food is selected from the group consisting of drinks, meats, chocolates, foods, confectionery, pizzas, ramen, other noodles, gums, candies, ice creams, alcohol drinks, vitamin complexes, and health supplement foods.

8. A method of preventing or treating metabolic syndrome in a mammal in need thereof, said method comprising: administering formic acid or a pharmaceutically acceptable salt thereof.

9. The method of claim 8, wherein the metabolic syndrome is selected from the group consisting of obesity, diabetes, arteriosclerosis, hypertension, hyperlipidemia, liver diseases, stroke, myocardial infarction, ischemic diseases, and cardiovascular diseases.
